(19) United States
(12) Patent Application Publication
(10) Pub. No.: US 2024/0108884 A1
(43) Pub. Date: Apr. 4, 2024

(54) ELECTRICAL CATHETER DEVICE, CATHETER, CABLE, AND METHOD FOR MANUFACTURING CATHETER

(71) Applicant: Japan Lifeline Co., Ltd., Tokyo (JP)

(72) Inventors: Takuya MASUDA, Tokyo (JP); Itsuki NAKAGAMI, Tokyo (JP)

(21) Appl. No.: 18/479,453

(22) Filed: Oct. 2, 2023

(30) Foreign Application Priority Data
Oct. 4, 2022 (JP) 2022-160112
Mar. 6, 2023 (JP) 2023-033832

(51) Int. Cl. A61N 1/05; A61N 1/39

(52) U.S. Cl. CPC A61N 1/0563; A61N 1/3925

(57) ABSTRACT

An electrical catheter device includes a catheter including a first electrode, a second electrode, a first conductor connected to the first electrode and extending toward a proximal end of a defibrillation catheter, a second conductor connected to the second electrode and extending toward the proximal end, and a connector that integrally accommodates a proximal end portion of the first conductor and a proximal end portion of the second conductor; and a voltage applying device that is connected to the connector and applies different voltages to the proximal end portion of the first conductor and the proximal end portion of the second conductor. In the connector, a creepage distance longer than a spatial distance is provided between the proximal end portion of the first conductor and the proximal end portion of the second conductor. [Selected Drawing] FIG. 3

### CROSS REFERENCE TO RELATED APPLICATIONS

This application claims the benefit of priority to Japanese Patent Application Numbers 2022-160112 and 2023-033832 filed on Oct. 4, 2022 and Mar. 6, 2023, respectively. The entire contents of each of the above-identified applications are hereby incorporated by reference.

### TECHNICAL FIELD

The present disclosure relates to an electrical catheter device, and the like.

### BACKGROUND

JP 05-115567 A discloses a defibrillation catheter that applies electrical stimulation to an arrhythmia site in the body. A distal end side of the defibrillation catheter is provided with two electrodes (group) to which a DC voltage is applied. A positive voltage (positive potential) is applied to one electrode, and a negative voltage (negative potential) is applied to the other electrode. In the present description, an electrode to which a positive voltage is applied at a certain point in time is referred to as a positive electrode or a +electrode for convenience, and an electrode to which a negative voltage is applied at the certain point in time is referred to as a negative electrode or a −electrode for convenience. At a point in time different from the certain point in time, a negative voltage may be applied to the positive electrode and a positive voltage may be applied to the negative electrode. However, at any point in time, different voltages are applied to the positive electrode and the negative electrode (or no voltage is applied to any electrode). Similarly, a conducting wire connected to the positive electrode and extending to a proximal end side of the catheter and a conducting wire connected to the negative electrode and extending to a proximal end side of the catheter are referred to as a positive electrode conducting wire and a negative electrode conducting wire, respectively, for convenience.

### SUMMARY

Because a high voltage that reaches 600 V, for example, may be applied between the positive electrode and the negative electrode, it is important to increase the insulation property between the positive electrode conducting wire and the negative electrode conducting wire so as not to cause short circuit. In defibrillation catheters of the related art, the positive electrode conducting wire and the negative electrode conducting wire may be completely isolated or separated for insulation purposes. In this case, a connector for connecting a proximal end portion of the defibrillation catheter to a cable of a voltage applying device has a bifurcated structure in which the connector is divided into a connector for the positive electrode conducting wire and a connector for the negative electrode conducting wire. Accordingly, the cable of the voltage applying device also has a bifurcated structure in which the cable is divided into a cable for connecting the positive electrode conducting wire and a cable for connecting the negative electrode conducting wire. Such a connector or cable having an increased volume or weight due to the complicated bifurcated structure may lower the operability of the defibrillation catheter. For example, if the cable becomes entangled along with an operation of the defibrillation catheter by a doctor, the defibrillation treatment may be interrupted in order to disentangle the entangled cable or to disconnect the cable from the connector and reconnect it.

The present disclosure has been made in view of such circumstances, and an object thereof is to provide an electrical catheter device and the like that can achieve balance between insulation property and operability.

In order to achieve the above object, according to an aspect of the present disclosure, there is provided an electrical catheter device including a catheter that includes a first electrode, a second electrode, a first conductor connected to the first electrode and extending toward a proximal end of the catheter, a second conductor connected to the second electrode and extending toward the proximal end, and a connector that integrally accommodates a proximal end portion of the first conductor and a proximal end portion of the second conductor; and a voltage applying device that is connected to the connector and applies different voltages to the proximal end portion of the first conductor and the proximal end portion of the second conductor. In the connector, a creepage distance is provided between the proximal end portion of the first conductor and the proximal end portion of the second conductor. The creepage distance is longer than a spatial distance between the proximal end portion of the first conductor and the proximal end portion of the second conductor.

In this aspect, because the connector integrally accommodates the proximal end portions of the first conductor (for example, electrically connected to the positive electrode conducting wire) and the second conductor (for example, electrically connected to the negative electrode conducting wire), the operability of the catheter can be improved as compared with the connector having the bifurcated structure described above. In addition, in the connector, because the creepage distance that is longer than the spatial distance between the proximal end portion of the first conductor and the proximal end portion of the second conductor is provided between the proximal end portion of the first conductor and the proximal end portion of the second conductor, the insulation property between both the conducting wires can also be enhanced.

Another aspect of the present disclosure relates to a catheter. The catheter includes a first electrode; a second electrode; a first conductor connected to the first electrode and extending toward a proximal end of the catheter; a second conductor connected to the second electrode and extending toward the proximal end; and a connector that is connected to a voltage applying device, the voltage applying device applying different voltages to a proximal end portion of the first conductor and a proximal end portion of the second conductor. The connector integrally accommodates the proximal end portion of the first conductor and the proximal end portion of the second conductor and is provided with a creepage distance between the proximal end portion of the first conductor and the proximal end portion of the second conductor. The creepage distance is longer than a spatial distance between the proximal end portion of the first conductor and the proximal end portion of the second conductor.

Still another aspect of the present disclosure relates to a cable. The cable includes a proximal end connected to a voltage applying device, the voltage applying device applying different voltages between a first electrode and a second electrode, the first electrode and the second electrode each provided in a catheter; and a distal end connected to a connector, the connector integrally accommodating a proximal end portion of a first conductor and a proximal end portion of a second conductor, the first conductor connected to the first electrode and extending toward a proximal end of the catheter, the second conductor connected to the second electrode and extending toward the proximal end. The connector is provided with a creepage distance between the proximal end portion of the first conductor and the proximal end portion of the second conductor. The creepage distance is longer than a spatial distance between the proximal end portion of the first conductor and the proximal end portion of the second conductor.

Yet another aspect of the present disclosure relates to a method for manufacturing a catheter. The method is a method for manufacturing a catheter including a first electrode, a second electrode, a first conducting wire connected to the first electrode and extending toward a proximal end of the catheter, a first terminal connected to a proximal end portion of the first conducting wire, a second conducting wire connected to the second electrode and extending toward the proximal end, a second terminal connected to a proximal end portion of the second conducting wire, and a connector that integrally accommodates the first terminal and the second terminal, the catheter provided with a creepage distance between a distal end portion of the first terminal and a distal end portion of the second terminal, the creepage distance longer than a spatial distance between the distal end portion of the first terminal and the distal end portion of the second terminal, the method including covering, with a second insulator, the distal end portion of the second terminal connected to the proximal end portion of the second conducting wire; and covering, with a first insulator, the distal end portion of the first terminal connected to the proximal end portion of the first conducting wire together with the second insulator.

Note that any combinations of the aforementioned components and those obtained by converting these expressions into methods, apparatuses, systems, recording media, computer programs, and the like are also included in the present disclosure.

According to the electrical catheter device and the like of the present disclosure, it is possible to achieve both insulation property and operability.

### BRIEF DESCRIPTION OF DRAWINGS

FIG. 1 illustrates an entire configuration of an electrical catheter device.

FIG. 2 is a cross-sectional view of a shaft 10 taken along a line A-A of FIG. 1.

FIG. 3 is a perspective view of a handle portion as seen from a proximal end side.

FIG. 4 schematically illustrates proximal end portions of a conductor group inside a connector body.

FIG. 5 illustrates a variation of arrangement of the proximal end portions of the conductor group as seen along an axial direction.

FIG. 6 illustrates a variation of the arrangement of the proximal end portions of the conductor group as seen along a radial direction.

FIG. 7 is a cross-sectional view illustrating a variation of a connector.

FIG. 8 schematically illustrates an insulator formed in two stages.

FIG. 9 schematically illustrates an insulator formed in two stages.

FIG. 10 schematically illustrates an insulator formed in two stages.

FIG. 11 schematically illustrates an insulator formed in two stages.

### DESCRIPTION OF EMBODIMENTS

Hereinafter, modes for carrying out the present disclosure (hereinafter, also referred to as embodiments) will be described in detail with reference to the drawings. In the description and/or drawings, the same or equivalent constituent elements, members, processing operations, and the like are denoted by the same reference numerals, and redundant descriptions are omitted. The scales and shapes of the illustrated parts are set for convenience to simplify the explanation and are not to be construed in a limited manner unless otherwise specified. The embodiments are illustrative and do not limit the scope of the present disclosure in any way. Not all features or combinations of the features described in the embodiments are essential to the present disclosure.

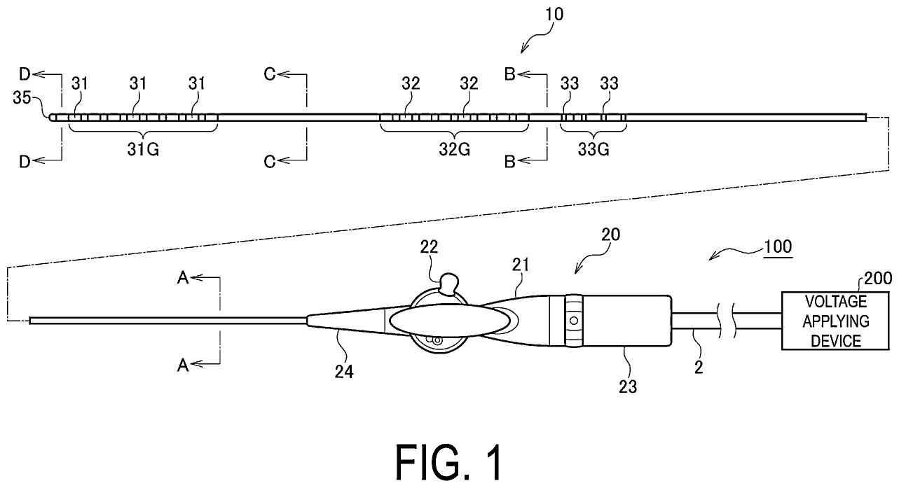

FIG. 1 illustrates an entire configuration of an electrical catheter device according to an embodiment of the present disclosure. The electrical catheter device includes a defibrillation catheter 100 and a voltage applying device 200. The defibrillation catheter 100 performs defibrillation by applying electrical stimulation to an arrhythmia site in the body. Typical examples of an arrhythmia site causing arrhythmia may include an atrium such as the right atrium and the left atrium, and a ventricle such as the right ventricle and the left ventricle (hereinafter, the atrium and the ventricle are also collectively referred to as a cardiac cavity). Note that the catheter that can be used for the electrical catheter device according to the present disclosure is not limited to the defibrillation catheter 100 whose target site is an inside of the cardiac cavity, which will be described in detail below. Specifically, the catheter may be any catheter as long as it can perform an electrical treatment on a target site in the body based on a DC or AC voltage between a plurality of electrodes (a first electrode and a second electrode described below) provided on a distal end side (inside the body). For example, a pulsed electric field ablation catheter that performs ablation processing on a target site such as an arrhythmia site by a pulsed electric field generated in response to a voltage between a plurality of electrodes may be used for the electrical catheter device according to the present disclosure.

The defibrillation catheter 100 includes a shaft 10 that is flexible, is inserted into the body, and has a tubular shape and a handle portion 20 connected to a proximal end side (outside the body) of the shaft 10. A doctor or the like who is an operator of the defibrillation catheter 100 operates the defibrillation catheter 100 while gripping the handle portion 20. The handle portion 20 includes a handle body 21, a knob-shaped operation portion 22, a connector 23, and a strain relief 24. The operator of the defibrillation catheter 100 can deflect (swing) a distal end portion (left end portion in FIG. 1) of the shaft 10 in a predetermined direction through a pull wire described below by rotating the operation portion 22 with a finger while gripping the handle body 21 with a hand. In addition, by rotating the handle body 21 in a circumferential direction, the distal end portion of the shaft 10 can be arranged in a desired direction. A distal end of a cable 2 of a voltage applying device 200 and/or an electrocardiograph is connected to the connector 23.

Three types of electrode groups of a first electrode group 31G, a second electrode group 32G and a third electrode group 33G are provided on a distal end side of the defibrillation catheter 100 and the shaft 10. The positions and order of the first electrode group 31G, the second electrode group 32G, and the third electrode group 33G in an axial direction of the shaft 10 (left-right direction in FIG. 1) can be freely selected, but in the illustrated example, the first electrode group 31G, the second electrode group 32G, and the third electrode group 33G are arranged in this order from the distal end toward the proximal end. In performing a defibrillation treatment on the inside of the cardiac cavity by the defibrillation catheter 100 having such an arrangement of the electrodes, for example, the first electrode group 31G on the distal end side is positioned at the coronary vein, the second electrode group 32G on the proximal end side is positioned at the right atrium, and the third electrode group 33G on the further proximal end side is positioned at the superior vena cava.

The first electrode group 31G includes a plurality of (eight, in the present embodiment) first electrodes 31 each having a ring shape and separated in the axial direction. The second electrode group 32G includes a plurality of (eight, in the present embodiment) second electrodes 32 each having a ring shape and separated in the axial direction. The third electrode group 33G includes a plurality of (four, in the present embodiment) third electrodes 33 each having a ring shape and separated in the axial direction. Note that each of the electrode groups 31G, 32G and 33G may be constituted by one electrode (this case is not strictly “an electrode group”). In addition, as in the illustrated example, the plurality of electrodes 31, 32 and 33 need not be collectively arranged for respective electrode groups 31G, 32G and 33G, but may also be arranged to be scattered in any order at any positions in the axial direction. A tip 35 for protection is provided at the distal end of the defibrillation catheter 100 and the shaft 10.

Voltages having different polarities are applied to the first electrode 31 and the second electrode 32 by the voltage applying device 200 connected to the connector 23. Specifically, a negative (−) voltage is applied to the second electrode 32 while a positive (+) voltage is applied to the first electrode 31, and a positive voltage is applied to the second electrode 32 while a negative voltage is applied to the first electrode 31. In addition, while neither a positive voltage nor a negative voltage is applied to the first electrode 31, neither a positive voltage nor a negative voltage is applied to the second electrode 32. In a typical defibrillation treatment, the defibrillation catheter 100 applies, to an arrhythmia site, electrical stimulation based on a DC voltage (applied by the voltage applying device 200 as a DC power supply device) between the first electrode 31 (for example, a coronary vein) and the second electrode 32 (right atrium).

Hereinafter, the first electrode 31 will be referred to as a positive electrode or a +electrode for convenience, and the second electrode 32 will be referred to as a negative electrode or a −electrode for convenience. This expression is based on only the fact that a positive voltage is applied to the first electrode 31 at a certain point in time and a negative voltage is applied to the second electrode 32 at the certain point in time. Thus, at a point in time different from the certain point in time, a negative voltage may be applied to the first electrode 31 as the positive electrode and a positive voltage may be applied to the second electrode 32 as the negative electrode. Note that it is also possible to apply an AC voltage between the first electrode 31 and the second electrode 32 by the voltage applying device 200 increasing a frequency of polarity reversal of the voltage that is applied to the first electrode 31 and the second electrode 32 (in this case, the voltage applying device 200 functions as an AC power supply device). As in the illustrated example, when the first electrode group 31G is constituted by the plurality of first electrodes 31 and the second electrode group 32G is constituted by the plurality of second electrodes 32, all the first electrodes 31 have substantially the same potential (for example, a positive potential) and all the second electrodes 32 have substantially the same potential (for example, a negative potential) having a polarity opposite to that of the first electrodes 31.

The third electrode 33 is provided on the distal end side of the defibrillation catheter 100 and the shaft 10 (in the example of the present embodiment, on a side closer to the proximal end than the first electrode 31 and the second electrode 32) and measures a potential (for example, a cardiac potential) in the body (for example, the superior vena cava where an abnormal potential is likely to occur). The third electrode 33 is connected to the voltage applying device 200 having functions of an electrocardiograph or the like, a stand-alone electrocardiograph or the like via the connector 23.

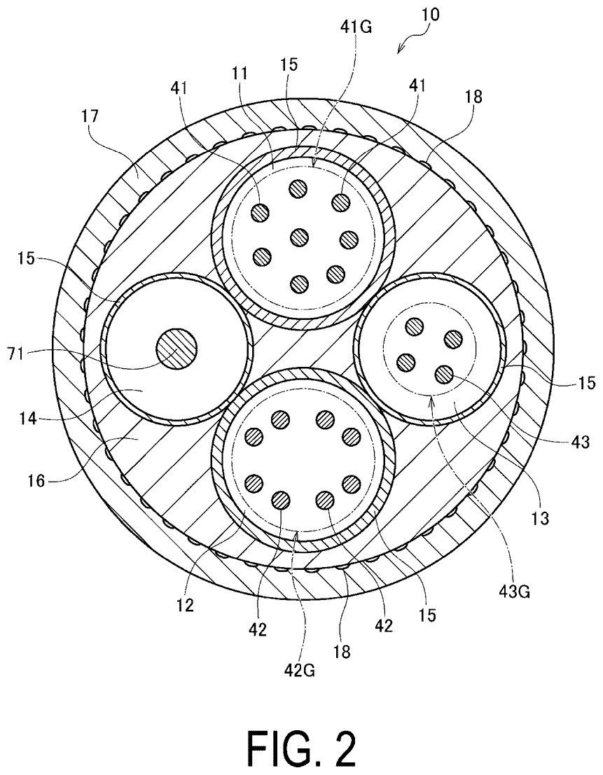

FIG. 2 is a cross-sectional view of the shaft 10 taken along a line A-A in FIG. 1. The A-A cross section is a cross section of the shaft 10 at a predetermined position on a side closer to the proximal end than any of the electrode groups 31G, 32G and 33G. Four lumens (inner cavities) of a first lumen 11, a second lumen 12, a third lumen 13, and a fourth lumen 14 are formed inside the shaft 10 having a tubular shape. Each of the lumens 11 to 14 is partitioned by an inner tube 15 having a tubular shape and made of a fluororesin or the like.

Inside the first lumen 11, a first conducting wire group 41G, which is electrically connected to the first electrode group 31G on the distal end side of the defibrillation catheter 100 and extends to the connector 23 on the proximal end side of the defibrillation catheter 100, is inserted. The first conducting wire group 41G includes the same number (eight, in the present embodiment) of first conducting wires 41 as the number of the plurality of first electrodes 31 in the first electrode group 31G. Distal end portions of the first conducting wires 41 are connected to the corresponding first electrodes 31 on a one-to-one basis. A proximal end portion of each of the first conducting wire 41 is connected to a distal end portion of the corresponding one of first terminals 41′ in a terminal portion 40 described below to form a first conductor 41″. A proximal end portion of each first conductor 41″ (i.e., proximal end portion of each first terminal 41′) is connected, in the connector 23, to the distal end of the cable 2 of the voltage applying device 200. With this structure, the first conducting wire group 41G and the first terminal group 41G′ connect the first electrode group 31G on the distal end side of the defibrillation catheter 100 and the voltage applying device 200 on the proximal end side of the defibrillation catheter 100 each other. The voltage applying device 200 can apply a positive voltage (or a negative voltage) to the first electrode group 31G as a positive electrode group through the cable 2 whose proximal end is connected to the voltage applying device 200, the first terminal group 41G′, and the first conducting wire group 41G.

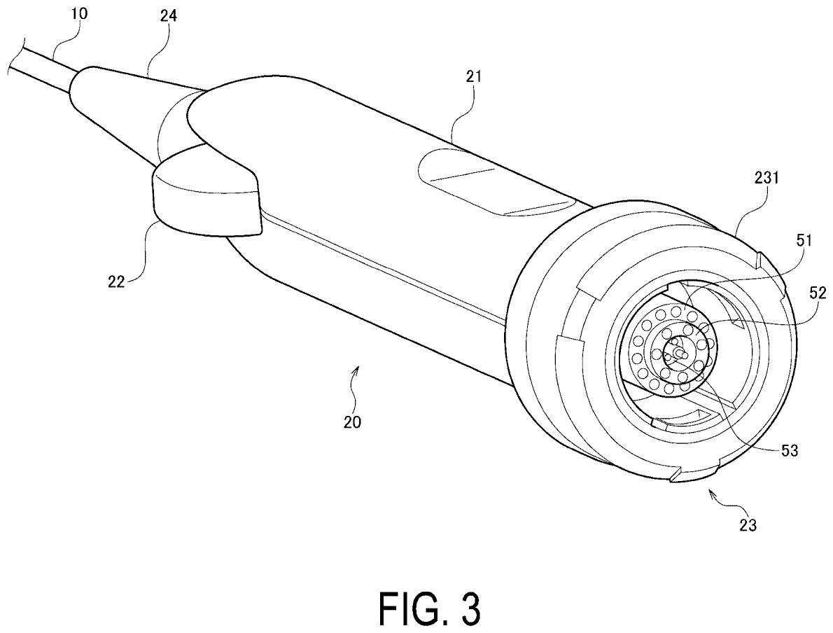

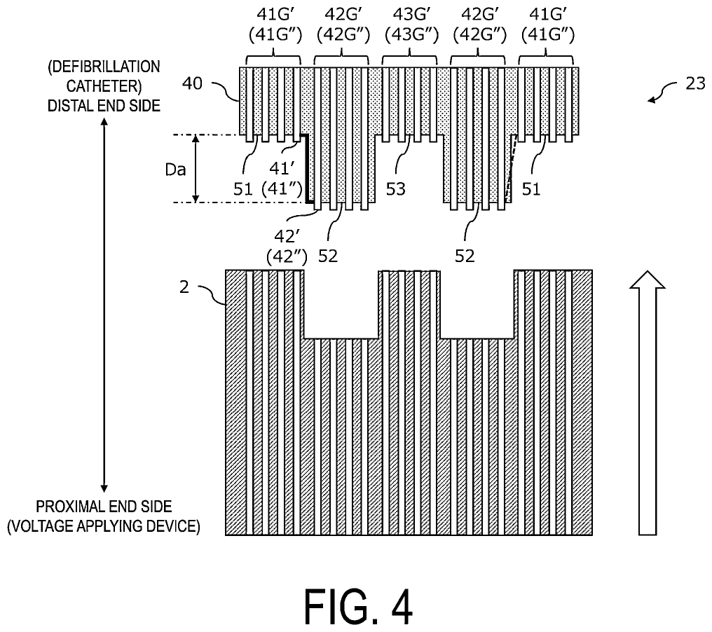

Note that the proximal end side of each of the first conducting wires 41 may extend to the inside of the connector 23 without interruption, but more practically, is electrically connected to the terminal portion 40 (a structure visible in the connector 23 (connector body 231) in FIG. 3, also refer to FIGS. 4 to 6) accommodated in the connector 23 on a distal end side (not illustrated) of the connector 23. In this case, what is accommodated in the connector 23 is not the proximal end portion of the first conducting wire 41 but the proximal end portion of the first terminal 41′ of the terminal portion 40 electrically connected to the proximal end portion of the first conducting wire 41. With this structure, the first conductor group 41G″ electrically connecting the first electrode group 31G and the connector 23 is constituted by the first conducting wire group 41G and the first terminal group 41G′ in the terminal portion 40, which are electrically connected to each other.

Inside the second lumen 12, a second conducting wire group 42G, which is electrically connected to the second electrode group 32G on the distal end side of the defibrillation catheter 100 and extends to the connector 23 on the proximal end side of the defibrillation catheter 100, is inserted. The second conducting wire group 42G includes the same number (eight, in the present embodiment) of second conducting wires 42 as the number of the plurality of second electrodes 32 in the second electrode group 32G. Distal end portions of the second conducting wires 42 are connected to the corresponding second electrodes 32 on a one-to-one basis. A proximal end portion of each of the second conducting wires 42 is connected to a distal end portion of the corresponding one of second terminals 42′ in the terminal portion 40 described below to form a second conductor 42″. A proximal end portion of each second conductor 42″ (i.e., proximal end portion of each second terminal 42′) is connected, in the connector 23, to the distal end of the cable 2 of the voltage applying device 200. With this structure, the second conducting wire group 42G and the second terminal group 42G′ connect the second electrode group 32G on the distal end side of the defibrillation catheter 100 and the voltage applying device 200 on the proximal end side of the defibrillation catheter 100 each other. The voltage applying device 200 can apply a negative voltage (or a positive voltage) to the second electrode group 32G as a negative electrode group through the cable 2 of the voltage applying device 200, the second terminal group 42G′, and the second conducting wire group 42G.

Note that the proximal end side of each of the second conducting wires 42 may extend to the inside of the connector 23 without interruption, but more practically, is electrically connected to the terminal portion 40 accommodated in the connector 23 on the distal end side (not illustrated) of the connector 23. In this case, what is accommodated in the connector 23 is not the proximal end portion of the second conducting wire 42 but the proximal end portion of the second terminal 42′ of the terminal portion 40 electrically connected to the proximal end portion of the second conducting wire 42. With this structure, the second conductor group 42G″ electrically connecting the second electrode group 32G and the connector 23 is constituted by the second conducting wire group 42G and the second terminal group 42G′ in the terminal portion 40, which are electrically connected to each other.

Inside the third lumen 13, a third conducting wire group 43G, which is electrically connected to the third electrode group 33G on the distal end side of the defibrillation catheter 100 and extends to the connector 23 on the proximal end side of the defibrillation catheter 100, is inserted. The third conducting wire group 43G includes the same number (four, in the present embodiment) of third conducting wires 43 as the number of the plurality of third electrodes 33 in the third electrode group 33G. Distal end portions of the third conducting wires 43 are connected to the corresponding third electrodes 33 on a one-to-one basis. A proximal end portion of each of the third conducting wires 43 is connected to a distal end portion of the corresponding one of third terminals 43′ in the terminal portion 40 described below to form a third conductor 43″. A proximal end portion of each third conductor 43″ (i.e., proximal end portion of each third terminal 43′) is connected, in the connector 23, to the distal end of the cable 2 of the voltage applying device 200 having functions of an electrocardiograph or a stand-alone electrocardiograph. With this structure, the third conducting wire group 43G and the third terminal group 43G′ connect the third electrode group 33G on the distal end side of the defibrillation catheter 100 and the voltage applying device 200 and/or the electrocardiograph on the proximal end side of the defibrillation catheter 100 each other. The voltage applying device 200 and/or the electrocardiograph can acquire the cardiac potential and the like measured by the third electrode group 33G as a measurement electrode group, through the cable 2 of the voltage applying device 200 and/or the electrocardiograph, the third terminal group 43G′ and the third conducting wire group 43G.

Note that the proximal end side of each of the third conducting wires 43 may extend to the inside of the connector 23 without interruption, but more practically, is electrically connected to the terminal portion 40 accommodated in the connector 23 on the distal end side (not illustrated) of the connector 23. In this case, what is accommodated in the connector 23 is not the proximal end portion of the third conducting wire 43 but the proximal end portion of the third terminal 43′ of the terminal portion 40 electrically connected to the proximal end portion of the third conducting wire 43. With this structure, the third conductor group 43G″ electrically connecting the third electrode group 33G and the connector 23 is constituted by the third conducting wire group 43G and the third terminal group 43G′ in the terminal portion 40, which are electrically connected to each other.

One pull wire 71 is inserted in the fourth lumen 14. A distal end portion of the pull wire 71 is fixed to the tip 35, and a proximal end portion of the pull wire 71 is fixed to the operation portion 22 of the handle portion 20. Because the fourth lumen 14 is displaced (eccentric) from a central axis of the shaft 10, the distal end portion of the shaft 10 can be deflected (swung) through an operation of the pull wire 71 by the operation portion 22. Specifically, when the operator of the defibrillation catheter 100 rotates the operation portion 22 in one direction, the pull wire 71 is pulled toward the proximal end (front in FIG. 2), and the distal end portion of the shaft 10 provided with the tip 35 is deflected in one direction (substantially leftward in FIG. 2). Similarly, when the operator of the defibrillation catheter 100 rotates the operation portion 22 in the other direction, the pull wire 71 is pushed out toward the distal end (back in FIG. 2), and the distal end portion of the shaft 10 provided with the tip 35 is deflected in the other direction (substantially rightward in FIG. 2).

FIG. 3 is a perspective view of the handle portion 20, as seen from a proximal end side. The connector 23 constituting the proximal end portion of the handle portion 20 includes a connector body 231 having a substantially cylindrical shape. The connector body 231 integrally accommodates a proximal end portion 51 of the first terminal group 41G′, a proximal end portion 52 of the second terminal group 42G′, and a proximal end portion 53 of the third terminal group 43G′. The distal end portion of the cable 2 of the voltage applying device 200 and/or the electrocardiograph is inserted into a space inside the connector body 231 from the proximal end side and can be attached to the terminal portion 40. The cable 2 includes three types of terminal groups corresponding to the first terminal group 41G′, the second terminal group 42G′, and the third terminal group 43G′ of the defibrillation catheter 100, and the distal end portion of each of the three types of terminal groups is connected to the corresponding one of the proximal end portions 51, 52, and 53 of the corresponding terminal groups 41G′, 42G′, and 43G′. Accordingly, the voltage applying device 200 can apply different voltages to the first electrode group 31G and the second electrode group 32G through the proximal end portion 51 of the first terminal group 41G′ and the proximal end portion 52 of the second terminal group 42G′. In addition, the voltage applying device 200 including functions of the electrocardiograph can acquire the cardiac potential measured by the third electrode group 33G through the proximal end portion 53 of the third terminal group 43G′.

FIG. 4 schematically illustrates the proximal end portions 51, 52, and 53 of the terminal groups 41G′, 42G′, and 43G′, respectively, of the terminal portion 40 inside the connector body 231. An upper part in this figure is the distal end side where the defibrillation catheter 100 is present, and a lower part in this figure is the proximal end side where the voltage applying device 200 is present. The proximal end portions 51, 52, and 53 of the terminal groups 41G′, 42G′, and 43G′, respectively, are schematically illustrated on the distal end side, and the cable 2 of the voltage applying device 200 including the three types of terminal groups corresponding to the terminal groups 41G′, 42G′, and 43G′ is schematically illustrated on the proximal end side. The distal end portion (upper end portion in FIG. 4) of the cable 2 is formed in an uneven shape conforming to an uneven shape formed by the proximal end portions 51, 52, and 53, which will be described below. For this reason, as schematically illustrated by an upward arrow in FIG. 4, the distal end portion of the cable 2 inserted toward the distal end side is fitted into the uneven shape of the proximal end portions 51, 52, and 53 and is thus firmly attached or fixed. When the cable 2 is connected to the connector 23, the proximal end portions 51, 52, and 53 may be slightly exposed.

Between the proximal end portion 51 of the first terminal group 41G′ as a positive electrode terminal group to which a positive voltage (or a negative voltage) is applied by the voltage applying device 200 and the proximal end portion 52 of the second terminal group 42G′ as a negative electrode terminal group to which a negative voltage (or a positive voltage) is applied by the voltage applying device 200, there is provided a creepage distance, which is longer than a spatial distance between the proximal end portions 51 and 52, for insulating both the proximal end portions 51 and 52. The creepage distance is a distance between conductors along a surface of an insulator. In the example of FIG. 4, a distance Da between a first end face on which the proximal end portion 51 of the first terminal group 41G′ is provided and a second end face on which the proximal end portion 52 of the second terminal group 42G′ is provided along a normal direction (up-down direction in FIG. 4) of at least one of the first end face or the second end face is the creepage distance between both the proximal end portions 51 and 52. Note that the creepage distance is not limited to an orthogonal plane orthogonal to the first end face and/or the second end face as illustrated in the drawings and may be provided on an intersection plane intersecting the first end face and/or the second end face. In addition, the creepage distance may be configured across surfaces of a plurality of different adjacent insulators.

Strictly speaking, as illustrated by a thick solid line on the left side in FIG. 4, the creepage distance between both the proximal end portions 51 and 52 is a distance along a surface of an insulator between the first terminal 41′ (the rightmost first terminal 41′ in the illustrated example) and the second terminal 42′ (the leftmost second terminal 42′ in the illustrated example) that are closest to each other among the plurality of first terminals 41′ (each of which is electrically connected to the corresponding one of the plurality of first conducting wires 41) constituting the first terminal group 41G′ and the plurality of second terminals 42′ (each of which is electrically connected to the corresponding one of the plurality of second conducting wires 42) constituting the second terminal group 42G′. Thus, the creepage distance between both the proximal end portions 51 and 52 slightly includes components in a direction (left-right direction in FIG. 4) orthogonal to the normal direction, in addition to the component Da in the normal direction. On the other hand, as illustrated by a thick dotted line on the right side in FIG. 4, the spatial distance between both the proximal end portions 51 and 52 is a distance obtained by straightly connecting positions of the first terminal 41′ and the second terminal 42′, which are closest to each other, slightly exposed from the connector 23 (insulator) and the cable 2 to be connected. Thus, a creepage distance obtained by connecting two points equivalent to these points by a polygonal line along the surface of the insulator is longer than a spatial distance obtained by straightly connecting the two points. Note that, in the following description, unless otherwise specified, the creepage distance between both the proximal end portions 51 and 52 is referred to as a distance Da in the normal direction.

As described above, the first end face (proximal end portion 51) and the second end face (proximal end portion 52), both of which have the axial direction (up-down direction in FIG. 4) as the normal direction, are arranged on different planes so as to form a step and/or unevenness having a height Da. In the illustrated example, the second end face further protrudes toward the proximal end side than the first end face by the creepage distance Da, but the first end face may be configured to further protrude toward the proximal end side than the second end face by the creepage distance Da.

Between the proximal end portion 51 of the first terminal group 41G′ and the proximal end portion 52 of the second terminal group 42G′, for example, a high voltage that reaches 600 V is applied by the voltage applying device 200. However, because both the proximal end portions 51 and 52 are insulated by the creepage distance Da, short circuit is effectively suppressed. In addition, because a spatial distance (thick dotted line) depending on the creepage distance Da is also formed between both the proximal end portions 51 and 52, dielectric breakdown is also effectively suppressed. Because such a creepage distance Da can be formed by a step and/or unevenness in one connector 23, it is not necessary to bifurcate the positive electrode terminal (first terminal 41′) and the negative electrode terminal (second terminal 42′) for insulation purposes as in the defibrillation catheter of the related art. As described above, according to the present embodiment, the proximal end portions 51 and 52 of the first terminal group 41G′ and the second terminal group 42G′, respectively, insulated by the creepage distance Da can be integrally and compactly accommodated in the single connector 23 and the connector body 231.

Accordingly, the connector 23 and the cable 2, which may have a bifurcated structure in the defibrillation catheter of the related art, have a simple single structure, and the operability of the defibrillation catheter 100 according to the present embodiment can be improved. In particular, in recent years, a defibrillation catheter is often inserted from a foot or the like that is relatively far from the heart, and thus, an operator such as a doctor is desired to perform a careful operation over a complicated and long route to the heart. According to the defibrillation catheter 100 of the present embodiment, because the connector 23 and the cable 2 have a single structure, the handling is facilitated, and thus, the possibility that the connector 23 and the cable 2 will be entangled during the operation is reduced. Therefore, it is possible to suppress a situation in which the defibrillation treatment is interrupted or the attention of the operator is distracted in order to deal with the problem of entanglement. In addition, although there is a possibility that a cleaning liquid or the like at the time of cleaning may enter the inside of the defibrillation catheter with the connector having a bifurcated structure of the related art, the connector 23 and the cable 2 having a single structure without bifurcation can suppress the liquid or the like from entering the defibrillation catheter.

As illustrated in FIG. 3, the proximal end portion 51 (first end face) of the first terminal group 41G′, the proximal end portion 52 (second end face) of the second terminal group 42G′, and the proximal end portion 53 (third end face) of the third terminal group 43G′ are arranged in a substantially concentric circle shape having the proximal end portion 53 as a center as seen along the axial direction of the defibrillation catheter 100. The first end face of the proximal end portion 51 positioned on the outermost side in the radial direction and the second end face of the proximal end portion 52 positioned on the inner side of the first end face are each formed in a substantially circular ring shape as seen along the axial direction. An inner circumference of the first end face and an outer circumference of the second end face substantially coincide with each other. With this structure, the first end face having a large diameter and a substantially circular ring shape surrounds the second end face having a small diameter and a substantially circular ring shape as seen along the axial direction (as seen along the normal direction of each end face in FIG. 4). In addition, the third end face of the proximal end portion 53 positioned on the innermost side in the radial direction is formed in a substantially circular shape as seen along the axial direction. The inner circumference of the second end face and an outer circumference of the third end face substantially coincide with each other. With this structure, the second end face having a substantially circular ring shape surrounds the third end face having a substantially circular shape as seen along the axial direction (as seen along the normal direction of each end face in FIG. 4).

As illustrated in FIGS. 3 and 4, between the proximal end portion 52 of the second terminal group 42G′ to which a positive voltage (or a negative voltage) is applied by the voltage applying device 200 and the proximal end portion 53 of the third terminal group 43G′ serving as a measurement terminal group from which cardiac potentials are acquired by the voltage applying device 200, a creepage distance, which is longer than a spatial distance between the proximal end portions 52 and 53, for insulating both the proximal end portions 52 and 53 may be provided. However, because the voltage between the second terminal group 42G′ and the third terminal group 43G′ is lower than the high voltage (for example, 600 V at maximum) between the first terminal group 41G′ and the second terminal group 42G′, the creepage distance between the proximal end portion 52 and the proximal end portion 53 may be less than the creepage distance Da between the proximal end portion 51 and the proximal end portion 52. In addition, the creepage distance between the proximal end portion 52 and the proximal end portion 53 may be substantially zero. In this case, the proximal end portion 52 and the proximal end portion 53 are arranged on the same plane (strictly, a slight creepage distance and a slight spatial distance are formed between the second terminal 42′ and the third terminal 43′ that are closest to each other).

A voltage (cardiac potential) whose absolute value is basically lower than those of the first terminal group 41G′ serving as the positive electrode terminal group and the second terminal group 42G′ serving as the negative electrode terminal group appears in the third terminal group 43G′. For this reason, the third terminal group 43G′ is less restricted in arrangement inside the connector 23 and the connector body 231 than the first terminal group 41G′ and the second terminal group 42G′ for which careful consideration is desired for ensuring insulation properties. Thus, in the example of FIG. 3, the connector 23 can be formed compact by efficiently arranging the proximal end portion 53 (third end face) of the third terminal group 43G′ in an empty space inside the proximal end portion 51 (first end face) of the first terminal group 41G′ and the proximal end portion 52 (second end face) of the second terminal group 42G′, both of which have the substantially circular ring shape.

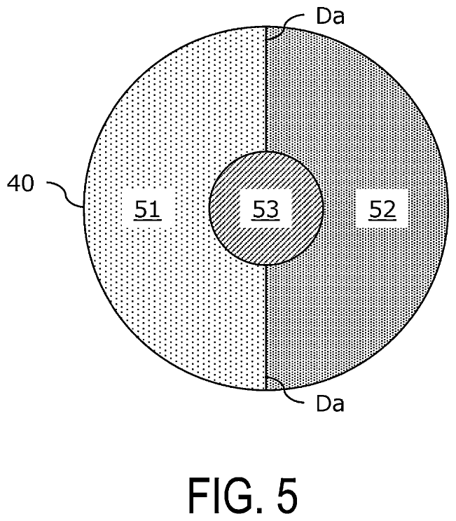

FIG. 5 illustrates a variation of the arrangement of the proximal end portions 51, 52, and 53 as seen along the axial direction. The proximal end portion 53 of the third terminal group 43G′ is arranged substantially at the center of the connector body 231, and the proximal end portion 51 of the first terminal group 41G′ and the proximal end portion 52 of the second terminal group 42G′ are arranged so as to sandwich the proximal end portion 53 from both sides. In the illustrated example, the third end face (proximal end portion 53) having a substantially circular shape is sandwiched between the first end face (proximal end portion 51) and the second end face (proximal end portion 52) each having a substantially semi-circular ring shape, so that a connection region of the cable 2 having a substantially circular shape as a whole is formed. A creepage distance Da in a direction perpendicular to the sheet surface in FIG. 5 is provided at a boundary between the proximal end portion 51 of the first terminal group 41G′ and the proximal end portion 52 of the second terminal group 42G′. Thus, one of the proximal end portions 51 and 52 is closer to the front (or back) of the sheet surface in FIG. 5 than the other. In addition, the proximal end portion 53 may be arranged on the same plane as the proximal end portions 51 or 52 or may be arranged on a plane different from the proximal end portions 51 and 52.

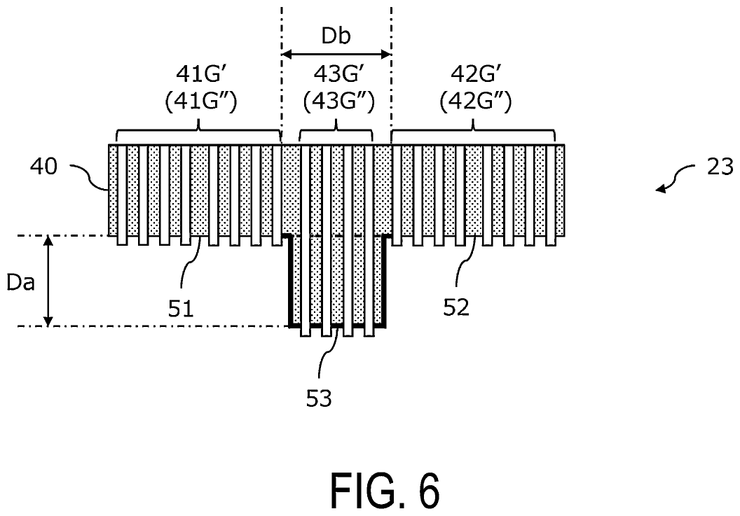

FIG. 6 illustrates a variation of the arrangement of the proximal end portions 51, 52, and 53 as seen along the radial direction. In this variation, between the proximal end portion 51 of the first terminal group 41G′ serving as a positive electrode terminal group to which a positive voltage (or a negative voltage) is applied by the voltage applying device 200 and the proximal end portion 52 of the second terminal group 42G′ serving as a negative electrode terminal group to which a negative voltage (or a positive voltage) is applied by the voltage applying device 200, a creepage distance in a direction intersecting the normal direction of at least one of the first end face (proximal end portion 51) or the second end face (proximal end portion 52) is provided. Specifically, in the example of FIG. 6, although the first end face of the proximal end portion 51 and the second end face of the proximal end portion 52 are arranged on the same plane, there are provided a creepage distance Db for insulating both the end faces in a direction (left-right direction in FIG. 6) orthogonal to the normal direction of the end faces, and a creepage distance Da similar to that in FIG. 4 for insulating both the end faces in the normal direction.

As illustrated by the thick solid line in FIG. 6, the creepage distance between both the proximal end portions 51 and 52 is a distance along a surface of an insulator between the first terminal 41′ (rightmost first terminal 41′ in the illustrated example) and the second terminal 42′ (leftmost second terminal 42′ in the illustrated example) that are closest to each other among the plurality of first terminals 41′ constituting the first terminal group 41G′ and the plurality of second terminals 42′ constituting the second terminal group 42G′. Specifically, the creepage distance between the proximal end portions 51 and 52 is a sum of a double (reciprocal distance) of the creepage distance Da in the normal direction and the creepage distance Db in the direction orthogonal to the normal direction. On the other hand, as illustrated by the thick dotted line in FIG. 6, the spatial distance between both the proximal end portions 51 and 52 is a distance Db obtained by straightly connecting the first terminal 41′ and the second terminal 42′ that are closest to each other. Thus, the creepage distance (2Da+Db) between both the proximal end portions 51 and 52 is longer than the spatial distance (Db) between both the proximal end portions 51 and 52.

In a region of the creepage distance Db in the left-right direction, the third terminal group 43G′ and the third end face (proximal end portion 53) may be provided as in the illustrated example, or nothing other than the insulator may be provided. In addition, a region of the creepage distance Db may protrude toward the proximal end side (lower side in FIG. 6) than the first end face (proximal end portion 51) and the second end face (proximal end portion 52) by the creepage distance Da as in the illustrated example or may be recessed toward the distal end side (upper side in FIG. 6) than the first end face and the second end face. As described above, the creepage distance between the first end face (proximal end portion 51) and the second end face (proximal end portion 52) may include components in both the axial direction (Da) and the radial direction (Db).

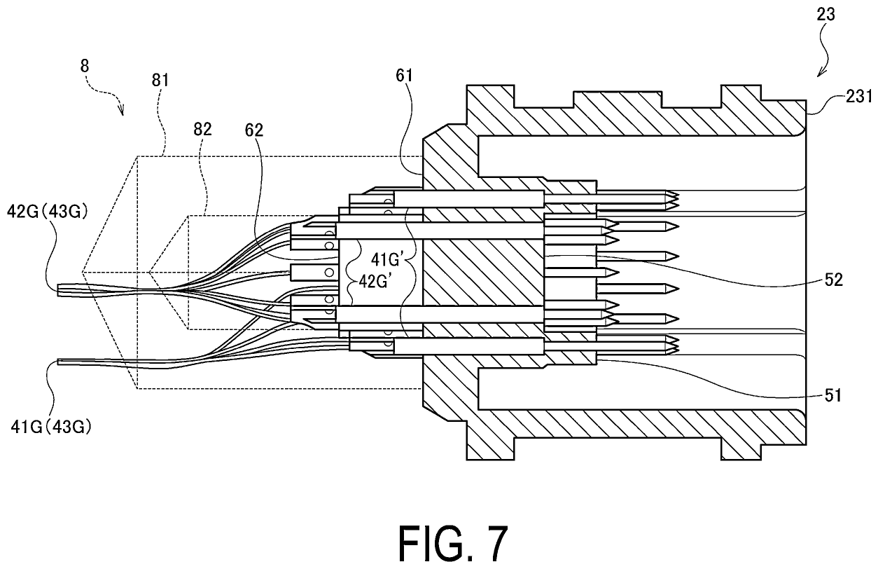

FIG. 7 is a cross-sectional view illustrating a variation of the connector 23. The left-right direction in this figure is the axial direction of the shaft 10, with the left side being the distal end side and the right side being the proximal end side. As described above, a space into which the cable 2 can be inserted is formed on the proximal end side of the connector body 231 of the connector 23.

In the examples illustrated in FIGS. 3 and 4, the third conducting wire group 43G and the third terminal group 43G′ (i.e., third conductor group 43G″) for acquiring the cardiac potential and the like measured by the third electrode group 33G are provided separately from the first conducting wire group 41G and the first terminal group 41G′ (i.e., first conductor group 41G″) and the second conducting wire group 42G and the second terminal group 42G′ (i.e., second conductor group 42G″). However, in the example of FIG. 7, the respective third conductors 43″ constituting the third conductor group 43G″ are incorporated into the first conductor group 41G″ and/or the second conductor group 42G″. For this reason, in FIG. 7, the first conductor group 41G″ (the first conducting wire group 41G and the first terminal group 41G′) and the second conductor group 42G″ (the second conducting wire group 42G and the second terminal group 42G′) are illustrated, for convenience, as representatives of the respective third conductors 43″ (the respective third conducting wires 43 and the respective third terminals 43′), as well, that can be included in each of the first conductor group and the second conductor group.

Similarly to the example of FIG. 4, between the proximal end portion 51 of the first terminal group 41G′ as a positive electrode terminal group to which a positive voltage (or a negative voltage) is applied by the voltage applying device 200 and the proximal end portion 52 of the second terminal group 42G′ as a negative electrode terminal group to which a negative voltage (or a positive voltage) is applied by the voltage applying device 200, there is provided a creepage distance, which is longer than a spatial distance between the proximal end portions 51 and 52, for insulating both the proximal end portions 51 and 52. Note that, there is a slight difference in that the proximal end portion 51 of the first terminal group 41G′ on the outer circumference side is recessed toward the distal end side and the proximal end portion 52 of the second terminal group 42G′ on the inner circumference side protrudes toward the proximal end side in the example of FIG. 4, while the proximal end portion 51 of the first terminal group 41G′ on the outer circumference side protrudes toward the proximal end side and the proximal end portion 52 of the second terminal group 42G′ on the inner circumference side is recessed toward the distal end side in the example of FIG. 7. However, because there is no difference between FIGS. 4 and 7 in that a creepage distance, which is longer than a spatial distance between the proximal end portion 51 and the proximal end portion 52, is provided between the proximal end portion 51 and the proximal end portion 52, also in the example of FIG. 7, the same action and/or effect specific to the present embodiment as in the example of FIG. 4 are achieved.

Further, in the example of FIG. 7, between a distal end portion 61 of the first terminal group 41G′ and a distal end portion 62 of the second terminal group 42G′, a creepage distance, which is longer than a spatial distance between the distal end portions 61 and 62, for insulating both the distal end portions 61 and 62 is also provided. Specifically, the distal end portion 61 of the first terminal group 41G′ on the outer circumference side is recessed toward the proximal end side, and the distal end portion 62 of the second terminal group 42G′ on the inner circumference side protrudes toward the distal end side. Note that even when the distal end portion 61 of the first terminal group 41G′ on the outer circumference side protrudes toward the distal end side and the distal end portion 62 of the second terminal group 42G′ on the inner circumference side is recessed toward the proximal end side, a creepage distance that achieves the same action and/or effect is provided.

As described above, on both the proximal end side (proximal end portions 51 and 52) and the distal end side (distal end portions 61 and 62) of the first terminal group 41G′ and the second terminal group 42G′, the creepage distance, which is longer than the spatial distance between the proximal end portions 51 and 52, for insulating both the proximal end portions 51 and 52 and the creepage distance, which is longer than the spatial distance between the distal end portions 61 and 62, for insulating both the distal end portions 61 and 62 are provided in the example of FIG. 7, and thus short circuit is effectively suppressed by the high insulation property even when a high voltage that reaches 600 V is applied by the voltage applying device 200. In addition, on both the proximal end side (proximal end portions 51 and 52) and the distal end side (distal end portions 61 and 62) of the first terminal group 41G′ and the second terminal group 42G′, the spatial distances depending on the corresponding creepage distances are also formed between the proximal end portions 51 and 52 and between the distal end portions 61 and 62, and thus dielectric breakdown is also effectively suppressed.

In the connector 23 constituting the proximal end portion of the shaft 10 or the entire defibrillation catheter 100 illustrated in FIG. 7, the proximal end portion of the first conducting wire group 41G connected to the first electrode group 31G at the distal end portion of the first conducting wire group 41G and extending to the proximal end side and the distal end portion of the first terminal group 41G′ integrally included in the connector 23 (connector body 231) are connected to each other, and the proximal end portion of the second conducting wire group 42G connected to the second electrode group 32G at the distal end portion of the second conducting wire group 42G and extending to the proximal end side and the distal end portion of the second terminal group 42G′ integrally included in the connector 23 (connector body 231) are connected to each other.

Here, the plurality of first conducting wires 41 and the plurality of second conducting wires 42 extending from the connector 23 toward the distal end side are each covered with a thin insulating film of polyamide-imide (PAI) or the like and are thus insulated from each other. However, in the present embodiment, for example, because a high voltage that reaches 600 V may be applied between the first conducting wire group 41G and the second conducting wire group 42G, it is preferable to further increase the insulation property between both the conducting wires. For this purpose, an insulator 8 formed of any insulating material such as polyamide is provided on the distal end side (left side in FIG. 7) of the connector 23.

The insulator 8 covers the proximal end portion of the first conducting wire group 41G and the proximal end portion of the second conducting wire group 42G so as to be insulated or isolated from each other. Furthermore, it is preferable that the insulator 8 covers not only the proximal end portion of the first conducting wire group 41G and the proximal end portion of the second conducting wire group 42G, but also the distal end portion of the first terminal group 41G′ to which the proximal end portion of the first conducting wire group 41G is connected and the distal end portion of the second terminal group 42G′ to which the proximal end portion of the second conducting wire group 42G is connected. For example, the insulator 8 substantially integrally molds the proximal end portion of the first conducting wire group 41G, the distal end portion 61 of the first terminal group 41G′, the proximal end portion of the second conducting wire group 42G, and the distal end portion 62 of the second terminal group 42G′.

In such a molded insulator 8, the first conducting wire group 41G and the second conducting wire group 42G are isolated from each other so as not to come into contact with each other. For example, as will be described below, by forming the insulator 8 in two stages of a first insulator 81 substantially molding only the first conducting wire group 41G and a second insulator 82 substantially molding only the second conducting wire group 42G, the first conducting wire group 41G and the second conducting wire group 42G can be effectively isolated in the insulator 8. However, the method for forming the insulator 8 can be freely selected as long as the first conducting wire group 41G and the second conducting wire group 42G are actually isolated from each other in the insulator 8.

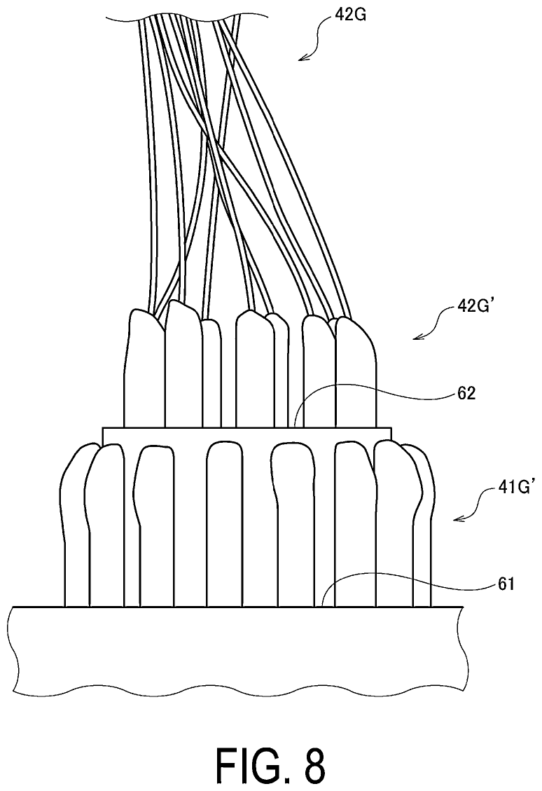

FIGS. 8 to 11 schematically illustrate the insulator 8 formed in two stages. FIG. 8 illustrates the distal end portion 61 of the first terminal group 41G′ and the distal end portion 62 of the second terminal group 42G′ before molding the insulator 8. As illustrated in this figure and other figures, the distal end portion 61 of the first terminal group 41G′ on the outer circumference side is provided closer to the proximal end side (lower side in FIG. 8) than the distal end portion 62 of the second terminal group 42G′ on the inner circumference side. In other words, the distal end portion 62 of the second terminal group 42G′ on the inner circumference side protrudes toward the distal end side (upper side in FIG. 8) than the distal end portion 61 of the first terminal group 41G′ on the outer circumference side. In addition, the distal end portion 61 of the first terminal group 41G′ on the outer circumference side surrounds the distal end portion 62 of the second terminal group 42G′ on the inner circumference side as seen along the axial direction (as seen along the up-down direction in FIG. 8).

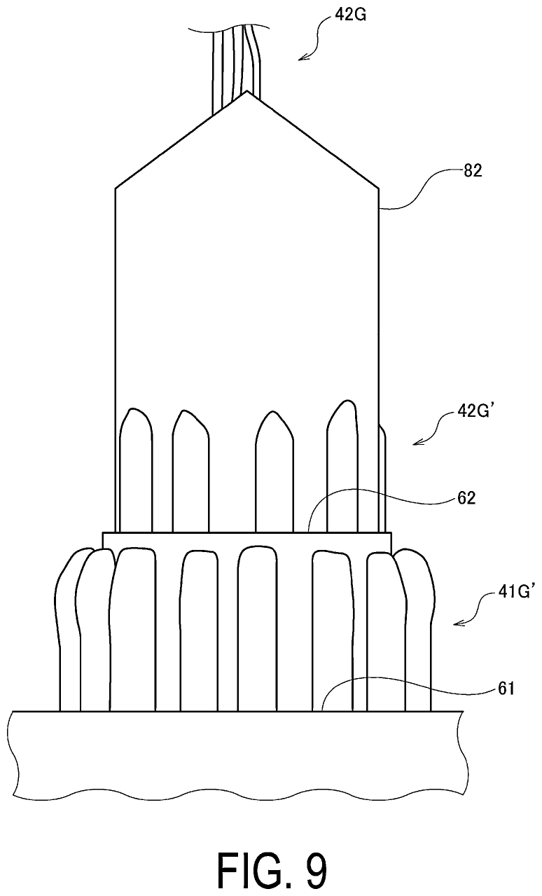

In the state illustrated in FIG. 8, the second conducting wire group 42G is connected only to the second terminal group 42G′ on the inner circumference side, and the first conducting wire group 41G is not yet connected to the first terminal group 41G′ on the outer circumference side. In subsequent FIG. 9, the distal end portion 62 of the second terminal group 42G′ to which the proximal end portion of the second conducting wire group 42G is connected is covered or molded with the second insulator 82 together with the second conducting wire group 42G. With this structure, the second insulator 82 substantially molds only the second conducting wire group 42G and the second terminal group 42G′ on the inner circumference side. The second conducting wire group 42G emerging from the distal end (upper end in FIG. 9) of the second insulator 82 toward the distal end side is bundled by an insulating tube made of polyimide or the like and is insulated or isolated from other members such as the first conducting wire group 41G.

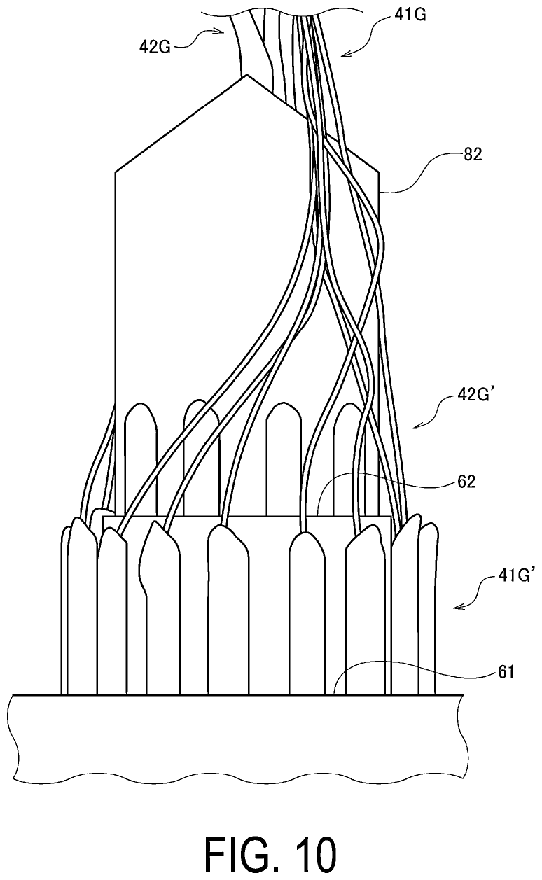

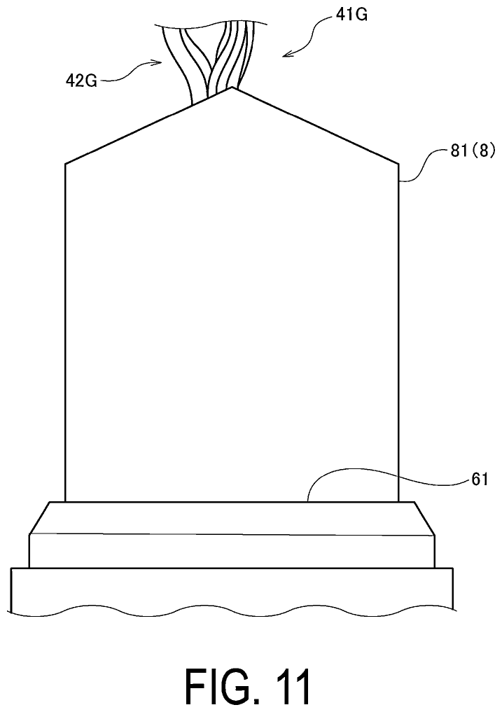

In FIG. 10 subsequent to FIG. 9, the first conducting wire group 41G is connected to the first terminal group 41G′ on the outer circumference side. Because the second conducting wire group 42G connected to the second terminal group 42G′ on the inner circumference side is molded by the second insulator 82, the second conducting wire group 42G is effectively insulated or isolated without coming into contact with the first conducting wire group 41G newly connected. In subsequent FIG. 11, the distal end portion 61 of the first terminal group 41G′ to which the proximal end portion of the first conducting wire group 41G is connected is covered or molded with the first insulator 81 together with the first conducting wire group 41G and the second insulator 82. As described above, the first insulator 81 substantially molds only the first conducting wire group 41G and the first terminal group 41G′ on the outer circumference side. The first conducting wire group 41G emerging from the distal end (upper end in FIG. 11) of the first insulator 81 (or the entire insulator 8) toward the distal end side is bundled by an insulating tube made of polyimide or the like and is insulated or isolated from other members such as the second conducting wire group 42G (bundled by another insulating tube).

As illustrated in FIG. 8, because the distal end portion 62 of the second terminal group 42G′ on the inner circumference side protrudes toward the distal end side than the distal end portion 61 of the first terminal group 41G′ on the outer circumference side, it is possible to easily form the second insulator 82 (FIG. 9) that substantially molds only the inner circumference side. Furthermore, it is possible to easily form the first insulator 81 (FIG. 11) on the outer circumference side, which is molded together with the second insulator 82 on the inner circumference side. Note that because the first insulator 81 and the second insulator 82 are formed in separate steps, a boundary between the first insulator 81 and the second insulator 82 can be detected by using any measurement technology such as an optical measurement technology as long as the boundary does not disappear due to heat or the like during formation.

In addition, similarly to FIG. 5, if the distal end portion 61 (corresponding to 51 in FIG. 5) of the first terminal group 41G′ and the distal end portion 62 (corresponding to 52 in FIG. 5) of the second terminal group 42G′ are arranged side by side as seen along the axial direction, desired insulation properties may be achieved by forming separately (or maybe simultaneously) the first insulator 81 for molding the distal end portion 61 and the second insulator 82 for molding the distal end portion 62.

The present disclosure has been described above based on the embodiments. It is obvious to those skilled in the art that various variations can be made to the combination of the components and processing operations in the exemplary embodiments and that such variations are included in the scope of the present disclosure.

Note that the configuration, action, and function of each device and each method described in the embodiments can be implemented by hardware resources, software resources or in cooperation of hardware resources and software resources. For example, processors, ROMs, RAMs, and various integrated circuits can be used as the hardware resources. For example, programs such as operating systems and applications can be used as the software resources.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. A catheter comprising:
a first electrode;
a second electrode;
a first conductor connected to the first electrode and extending toward a proximal end of the catheter;
a second conductor connected to the second electrode and extending toward the proximal end, and
a connector connected to a voltage applying device, the voltage applying device configured to apply different voltages to a proximal end portion of the first conductor and a proximal end portion of the second conductor, the connector configured to integrally accommodate the proximal end portion of the first conductor and the proximal end portion of the second conductor and provided with a creepage distance between the proximal end portion of the first conductor and the proximal end portion of the second conductor, the creepage distance longer than a spatial distance between the proximal end portion of the first conductor and the proximal end portion of the second conductor.

2. The catheter according to claim 1, wherein the creepage distance is provided on an intersection plane intersecting a first end surface where the proximal end portion of the first conductor is provided.

3. The catheter according to claim 2, wherein the intersection plane intersects a second end face where the proximal end portion of the second conductor is provided, and
wherein the first end face surrounds the second end face as seen along a normal direction of at least one of the first end face or the second end face.

4. The catheter according to claim 3,
wherein the connector integrally accommodates, together with the proximal end portion of the first conductor and the proximal end portion of the second conductor, a proximal end portion of a third conductor that is connected to a third electrode provided in the catheter and configured to measure a potential in a body and is extends toward the proximal end, and
wherein the second end face surrounds a third end face where the proximal end portion of the third conductor is provided as seen along the normal direction.

5. The catheter according to claim 1, wherein in the connector, between a first end face where the proximal end portion of the first conductor is provided and a second end face where the proximal end portion of the second conductor is provided, the creepage distance is provided in a direction intersecting a normal direction of at least one of the first end face or the second end face.

6. The catheter according to claim 1,
wherein the first conductor includes a first conducting wire connected to the first electrode and extending to the proximal end portion of the first conductor, and a first terminal connected to the first conducting wire at the proximal end portion of the first conductor,
wherein the second conductor includes a second conducting wire connected to the second electrode and extending to the proximal end portion of the second conductor, and a second terminal connected to the second conducting wire at the proximal end portion of the second conductor,
wherein the connector integrally accommodates the first terminal and the second terminal, and
wherein the creepage distance is provided between the first terminal and the second terminal.

7. The catheter according to claim 6, wherein the creepage distance is provided between the proximal end portion of the first terminal and the proximal end portion of the second terminal.

8. The catheter according to claim 6, wherein the creepage distance is provided between a distal end portion of the first terminal and a distal end portion of the second terminal.

9. The catheter according to claim 8, further comprising an insulator configured to cover a proximal end portion of the first conducting wire and a proximal end portion of the second conducting wire to insulate the proximal end portion of the first conducting wire and the proximal end portion of the second conducting wire from each other.

10. The catheter according to claim 9, wherein the insulator includes a second insulator configured to cover the distal end portion of the second terminal connected to the proximal end portion of the second conducting wire, and a first insulator configured to cover, together with the second insulator, the distal end portion of the first terminal connected to the proximal end portion of the first conducting wire.

11. The catheter according to claim 10, wherein the first terminal is provided on an outer peripheral side of the second terminal as seen along an axial direction where at least one of the first conducting wire or the second conducting wire extends.

12. The catheter according to claim 11, wherein the distal end portion of the first terminal is provided closer to the proximal end than the distal end portion of the second terminal.

13. The catheter according to claim 1, wherein the catheter is a defibrillation catheter configured to apply, to an arrhythmia site, electrical stimulation based on a voltage between the first electrode and the second electrode.

14. A cable comprising:
a proximal end connected to a voltage applying device, the voltage applying device configured to apply different voltages between a first electrode and a second electrode, the first electrode and the second electrode each provided in a catheter; and
a distal end connected to a connector, the connector configured to integrally accommodate a proximal end portion of a first conductor and a proximal end portion of a second conductor, the first conductor connected to the first electrode and extending toward a proximal end of the catheter, the second conductor connected to the second electrode and extending toward the proximal end,
wherein the connector is provided with a creepage distance between the proximal end portion of the first conductor and the proximal end portion of the second conductor, the creepage distance longer than a spatial distance between the proximal end portion of the first conductor and the proximal end portion of the second conductor.

15. A method for manufacturing a catheter including a first electrode, a second electrode, a first conducting wire connected to the first electrode and extending toward a proximal end of the catheter, a first terminal connected to a proximal end portion of the first conducting wire, a second conducting wire connected to the second electrode and extending toward the proximal end, a second terminal connected to a proximal end portion of the second conducting wire, and a connector configured to integrally accommodate the first terminal and the second terminal, the catheter provided with a creepage distance between a distal end portion of the first terminal and a distal end portion of the second terminal, the creepage distance longer than a spatial distance between the distal end portion of the first terminal and the distal end portion of the second terminal, the method comprising:
covering, with a second insulator, the distal end portion of the second terminal connected to the proximal end portion of the second conducting wire; and
covering, with a first insulator, the distal end portion of the first terminal connected to the proximal end portion of the first conducting wire, together with the second insulator.
